(19) United States
(12) Patent Application Publication
(10) Pub. No.: US 2010/0069850 A1
(43) Pub. Date: Mar. 18, 2010

(54) WOUND DRESSING HAVING PRESSURE-DISTRIBUTING TUBE INLET

(75) Inventor: Tomas Fabo, Molnlycke (SE)

Correspondence Address: YOUNG & THOMPSON, 209 Madison Street, Suite 500, Alexandria, VA, 22314, US

(73) Assignee: MOLNLYCKE HEALTH CARE AB, Goteborg (SE)

(21) Appl. No.: 12/440,854

(22) PCT Filed: Sep. 20, 2007
(86) PCT No.: PCT/SE07/50668

§ 371 Date: May 7, 2009

(30) Foreign Application Priority Data
Oct. 3, 2006 (SE) 0602064-8

(51) Int. Cl. A61M 5/31; A61F 13/00

(52) U.S. Cl. 604/180; 602/52

(57) ABSTRACT

A wound dressing includes a tube (9) for evacuating fluid from a wound and an air-tight and liquid-tight envelope layer (10) of flexible material, which envelope layer has a central region, in which the tube emerges beneath the envelope layer, and a peripheral region, which extends beyond the central region, the tube from the central region running outwards into the peripheral region beneath the envelope layer and out from this. The tube (9) is enclosed in a pressure-distributing material piece (11) which surrounds the tube, extends along the tube in the peripheral region, and also extends laterally beyond the tube viewed in the longitudinal direction of the tube.

### TECHNICAL FIELD

The present invention relates to a wound dressing comprising a tube for evacuating fluid from a wound and an air-tight and liquid-tight envelope layer of flexible material, which envelope layer has a central region, in which the tube emerges beneath the envelope layer, and a peripheral region, which extends beyond the central region, the tube from the central region running outwards into the peripheral region beneath the envelope layer and out from this.

### BACKGROUND ART

Wound dressings of the above-stated type are used for, with the aid of underpressure, sucking up fluid from a wound or for supplying fluid to a wound via one or more tubes. The treatment of wounds with the aid of underpressure has been shown to accelerate the healing of the wound, so that there is a need to create effective and easily applied dressings with which it is easy, by simple means, to maintain the desired underpressure in the wound region.

To prevent the tube or tubes in such dressings from collapsing owing to the pressure difference between the underpressure in the wound region and the atmospheric pressure, they must have a certain hardness. Moreover, these tubes run outwards from the edge of the wound and are therefore situated in a region in which the skin of a patient is especially sensitive to external influence. There is therefore a risk that the tubes, in the event of external load, for example pressure bandaging or the patient's own weight, give rise to a damaging local load upon the sensitive skin or tissue next to the wound.

Another problem is to achieve a seal-tight inlet for the tubes in the peripheral region beyond a wound, so that fluid cannot leak out beyond the dressing and so that the underpressure in the wound region is not jeopardized. Even small leakage paths can make it difficult to maintain the desired underpressure or can require complicated control apparatus to keep the underpressure within given limits.

The object of the present invention is to solve these problems and to produce a wound dressing of the type stated in the introduction, in which the risk of tubes being able to harm a patient as a result of local load is slight and in which each tube inlet is seal-tight.

### DISCLOSURE OF INVENTION

These objects are achieved according to the invention with a wound dressing comprising a tube for evacuating fluid from a wound and an air-tight and liquid-tight envelope layer of flexible material, which envelope layer has a central region, in which the tube emerges beneath the envelope layer, and a peripheral region, which extends beyond the central region, the tube from the central region running outwards into the peripheral region beneath the envelope layer and out from this, characterized in that the tube is enclosed in a pressure-distributing material piece which surrounds the tube, extends along the tube in the peripheral region, and also extends laterally beyond the tube viewed in the longitudinal direction of the tube. As a result of the pressure-distributing material piece, any external load upon that part of the dressing which contains the tubes is distributed to that surface of the whole of the material piece which bears against the skin or tissue of the patient. This surface is considerably larger than the bottommost part of the tube or tubes, which would otherwise bring load to bear upon the underlying skin or tissue of the patient. By distributing the load over a large area, the risk of damage to skin or tissue becomes very small. Moreover, as a result of the pressure-distributing material piece, an, at least on a macroscale, plane surface is produced, which can easily be sealingly fixed to the skin, either by being intrinsically adherent to skin or by being provided with an adhesive which is adherent to skin.

In a preferred embodiment, the pressure-distributing material piece has in cross section a shape which tapers off from the tube in the lateral direction. The pressure-distributing material piece advantageously consists of a silicone composition, preferably an addition-curing RTV silicone system. Moreover, the material piece is preferably adherent to skin. In an advantageous variant, the material piece is shaped in situ. In such a variant, the material piece per se ensures both adhesion to and sealing against underlying skin.

A tube can be provided for the supply of fluid to a wound and can be enclosed in a pressure-distributing material piece, the tubes for the evacuation and supply of fluid running alongside each other and preferably being enclosed in the same material piece.

The material piece has preferably a hardness of 2-15 mm.

### BRIEF DESCRIPTION OF DRAWINGS

The invention will now be described with reference to the appended figures, whereof:

FIG. 1 shows diagrammatically a cross-sectional view of a first embodiment of a wound dressing according to the invention,

FIG. 2 shows a plan view, partly sectioned, of the wound dressing in FIG. 1,

FIG. 3 shows a cross-sectional view along the line in FIG. 2,

FIG. 4 shows diagrammatically the irrigation dressing according to FIG. 1 applied to a patient,

FIG. 5 illustrates diagrammatically a wound dressing according to a second embodiment of the invention, and

FIGS. 6 and 7 illustrates a method of measuring softness (hardness).

### MODE(S) FOR CARRYING OUT THE INVENTION

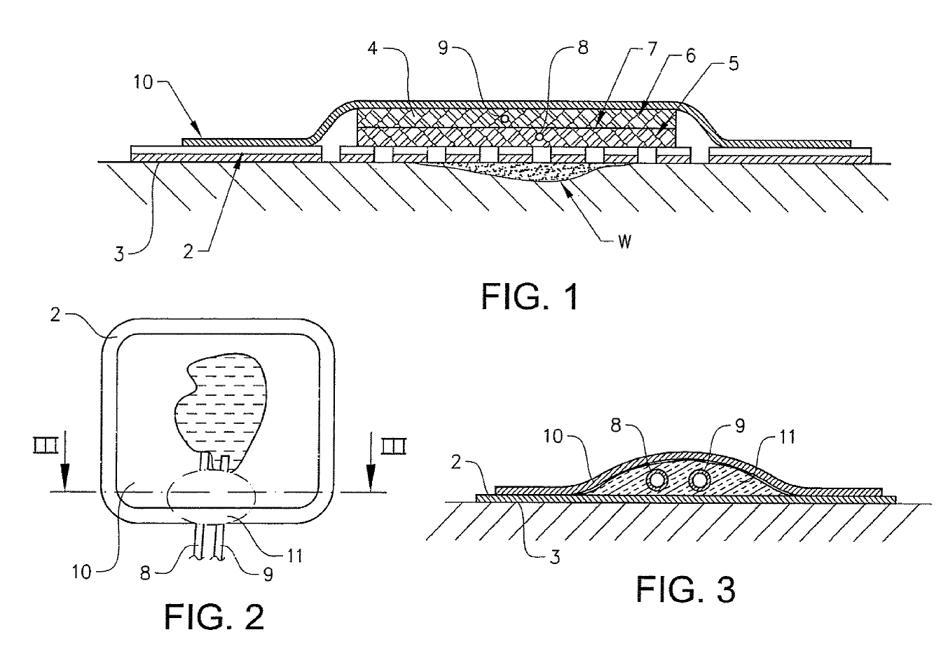

FIG. 1 shows in diagrammatic representation a wound dressing according to a first, preferred embodiment of the invention applied over a wound W of a patient P. The first component of the dressing is constituted by a film 2 coated with a coating 3 of an adhesive, which preferably seals against microleakage.

As can be seen from FIG. 1, the film 2 and its adhesive coating 3 are perforated within a central region extending over and around the wound W.

A main function of the adhesive coating 3 is to connect the dressing 1 in a seal-tight manner to the skin of the patent so that air is prevented from leaking in between the skin and the adhesive coating, and to securely fix the dressing to the skin so that the product remains in place under all normal loads to which the dressing is subjected.

Moreover, the adhesive in the coating must be skin-friendly and allow the dressings to be removed without damaging the skin.

The adhesive can advantageously be constituted by a silicone elastomer which is very soft and has low surface energy so that it wets very well against the skin. i.e. it flows out into the irregularities in the skin and creates a large contact surface between skin and silicone elastomer. This large contact surface helps to make the silicone elastomer attach well to the skin, despite the fact that the binding force of the silicone elastomer against skin is not intrinsically very strong. The adhesive strength constitutes a measure of the energy which is required to separate and pull off the adhesive layer from the skin. A contributory factor to the need for high energy and hence high pull-off force to remove the silicone elastomer from the skin despite the relatively weak binding force is that a great deal of energy is expended in stretching the soft silicone elastomer before it comes loose from the skin. The softer and thicker the layer of silicone elastomer, the more force/energy is expended to remove the elastomer from the skin.

Since the characteristics of the skin vary from person to person, the adherence of the adhesive coating to the skin also, of course, varies for different patients. The adherence is also dependent on the thickness of the soft adhesive and the mechanical properties of the substrate.

Adhesives which are usable with film dressings according to the invention must have an adhesive strength of at least 0.2-4 N/25 mm measured using a method developed by the Applicant, which, inter alia, is described in WO 2006/075949 A1. Preferably, the adhesive strength is 1-2.5 N/25 mm.

Adhesives according to the present invention must have a softness that exceeds 10 mm measured by a method based on ASTM D 937 and ASTM D 51580. Certain modifications have been made and are described below. FIGS. 6 and 7 illustrate this modified method for measuring softness of an adhesive by letting a cone B with a weight of 62.5 g penetrate by gravity into a 30 mm thick sample C of the adhesive whose softness is to be determined. The sample is produced by filling a cylindrical glass container, which has an internal diameter of 60 mm and an internal height of 35-40 mm, with adhesive up to a height of 30 mm. For a silicone elastomer, non-cured silicone prepolymer is poured into the container and is then crosslinked to an elastomer in the glass cylinder. The cone that is used is shown in FIG. 6 and has the following dimensions: a=65 mm, b=30 mm, c=15 mm and d=8.5 mm. When carrying out the method for measuring softness, the cone B is first lowered to a position 1, which is shown by broken lines in FIG. 7 and in which the tip of the cone just brushes the surface of the sample C. The cone B is then released, such that it can penetrate into the sample C by force of gravity. The number of mm by which the tip of the cone B has penetrated into the sample C after 5 seconds is measured and constitutes the penetration value P, which is greater, the softer the sample. The penetration value P constitutes the measurement of softness used in the present invention. The method is carried out using a Penetrometer PNR 10 from Sommer & Runge KG, Germany.

It has been shown that even in the case of soft, skin-friendly adhesives, which form barriers preventing liquid flow through them, liquid and air can leak through these barriers via skin cracks, skin creases or other irregularities in the skin. In an examination of leak-proofness from film dressings, the Applicant discovered an unexpected weakness in the standard film dressings. In studies under a microscope, it has been shown that liquids can easily be spread beneath the film dressings, despite the fact that they appear to be firmly stuck with a fully tight seal against the skin. Liquid has proved to be able to spread several centimetres beneath the dressings via the naturally occurring microscopic creases on normal skin. Since the leakage is constituted by very small quantities and is not visible if the infiltration of uncoloured liquids is studied, this has previously been overlooked. The phenomenon, referred to as microleakage, could only be observed once the liquid was dyed with strongly coloured colouring pigment.

It has surprisingly been shown that for a skin-friendly adhesive, the abovementioned leakage risk can be eliminated, or at least significantly reduced, if the adhesive is sufficiently soft and has a sufficiently high weight per unit area. It has also been shown that such adhesives also prevent leakage of air through the adhesive barrier between dressing and skin. A correlation has been shown to exist between softness (penetration) and weight per unit area of the silicone elastomer. The softer the silicone elastomer, the less weight per unit area is required for sealing. The correlation between weight per unit area and softness is such that, in order to achieve seal-tightness at low weights per unit area, very soft adhesives are required, whilst less soft adhesives require higher weights per unit area to achieve seal-tightness. It has been shown that, at softnesses less than 10 mm, it is difficult, perhaps impossible, to achieve seal-tight film dressings. At softnesses around 20 mm, a weight per unit area of 50 g/m² may be sufficient to obtain sealing. It has been shown that the soft adhesives which seal against microleakage also seal against air leakage.

Apart from increasing the leak-proofness, a higher weight per unit area of the adhesive coating also gives a reduced risk of blisters, pimples or other damage arising from movements of the wearer of the film dressing which lead to relative movement between skin and adhesive coating, or caused by the dressing being subjected to load by external forces, for example if the wearer of the dressing bangs against an object. It has been shown that the risk of occurrence of such damage diminishes with higher weight per unit area and higher softness of the adhesive coating. This is probably due to the fact a part of the load is absorbed by the adhesive layer through deformation and hence is not transmitted to the skin. Moreover, the dressing according to the invention can stretch together with the skin, which reduces the risk of shearing occurring between skin and adhesive, which can give rise to mechanical damage to the skin.

In order to allow only a small application force to be needed in the application of dressings according to the present invention, the softness of the soft, skin-friendly adhesive which is used is preferably greater than 10 mm, particularly preferably lying within the range 12-17 mm. The softer an adhesive is, the faster it flows into any irregularities in the support surface, which means that the dressings according to the present invention are leak-proof against both liquid and air directly after being applied to normal skin. If the softness is greater than 17 mm, there is a risk that the inner cohesion of the adhesive will be too small, so that remnants of adhesive are left behind on the skin when an applied dressing is removed.

Another important characteristic is that the adhesive strength of the soft, skin-friendly adhesives which are used in dressings according to the invention does not alter over time, or alters over time only to a small degree. This ensures that the dressing is kind to the skin, for example newly formed, delicate skin, even when a dressing which has sat on a patient for a long time is removed.

The adhesive layer 3 is advantageously made up of a silicone composition, which, after mixing together, cross-links into a soft elastomer. Especially suitable are RTV (Room Temperature Vulcanizing) silicone systems, which are addition-curing and which can be cross-linked at moderate temperatures, RTV silicones can be made soft, pliable and self-adhesive.

Examples of RTV addition-curing silicone systems are quoted in EP 0 300 620 A1, in which so-called “gel-forming compositions”, consisting of an alkenyl-substituted polydiorganosiloxane, an organosiloxane containing hydrogen atoms bound to a part of the silicone atoms, and a platinum catalyst, are described.

Examples of a commercially available RTV silicone system are Wacker SilGel 612 from Wacker-Chemie GmbH, Munich, Germany. This is a 2-components system. By varying the proportions between the two components A:B from 1.0:0.7 to 1.0:1.3, the softness and the adhesion level of the formed elastomer can be varied.

Examples of further soft silicone elastomers which are adherent to dry skin are NuSil MED-6340, NuSil MEDS-6300, NuSil MED12-6300 from NuSil Technology, Carpinteria, Ga., USA and Dow Corning 7-9800 from Dow Corning Corporation, Midland, USA.

It is also conceivable to use soft hot-melt adhesive. As an example of this type of adhesive, Dispomel 70-4647 from National Starch, USA can be cited.

In the embodiment shown in FIG. 1, there are on top of the film 2 a component 4 consisting of two bodies 5, 6 made of a soft fluid-distributing material, preferably a cellular material, for example polyurethane foam, a membrane 7, disposed between these bodies 5, 6, made of an airtight and liquid-tight material, and two tubes 8, 9 for the supply and evacuation, respectively, of fluid. The supply tube 8 emerges beneath the membrane 7 and the evacuation tube 9 emerges above the membrane. The terms “above”, “below” and similar position specifications in the description and patent claims are relative and refer to FIG. 1, in which the dressing is placed on the top side of a patient. The relative positions of the components in the dressing are the same, even if the dressing were to be placed on, for example, the underside of an arm of a patient.

On top of the component 4 there extends an envelope layer 10, preferably a plastics film, for example of polyurethane plastic. The envelope layer 10 extends over the whole of the component 4 and also beyond its contour. That part of the envelope layer 10 which extends beyond the contour of the component 4 is sealingly fixed to the top side of the film 2, for example by gluing or heat sealing.

As can be seen from FIG. 2, the tubes 8, 9 run alongside each other between the envelope layer 10 and the plastics film 2 upon exit from the cellular bodies 5, 6. According to the invention, the tubes 8, 9 are enclosed in a pressure-distributing material piece 11 in the region between the edge of the cellular bodies and the outer edge of the dressing. The material piece 11 has the shape of a disc which tapers off in the lateral direction relative to the longitudinal direction of the tubes 8, 9 and in the direction away from the tubes. The material piece 11 is therefore thickest in those parts which are located close to the tubes. Moreover, the material piece is made of a material which has a hardness such that, when subjected to an external load, the forces will be distributed in the material piece and will bring load to bear upon underlying skin and tissue spread over a relatively large plane surface. The presence of the material serves to prevent a force on the top side of a tube from being transmitted as a linear load, which could damage underlying skin and tissue. Since the tubes must be able to withstand an underpressure in the wound region without collapsing, they have a relatively large deformation resistance. Preferably, the deformation resistance of tubes and material piece is equal in magnitude. The material piece 11 should have a hardness of 2-15 mm, preferably 3-13 mm measured by the same modified method based on ASTM D 937 and ASTM D 51580 used for measuring softness of adhesives.

To prevent liquid from being able to force its way out of the dressing or air from forcing its way in between the bottom side of the material piece 11 and the top side of the plastics film 2, the material piece 11 is fixed to the plastics film 2 in a suitable manner, for example by gluing, or by the material piece per se being made of a material which adheres to the plastics film.

It is pointed out that, from the pressure distribution aspect, it is not necessary for the tubes to be wholly enclosed in the material piece 11, but rather it is sufficient for their lower halves to be embedded in the material piece. It is expedient, however, for the material piece also to cover the greater part of the upper halves of the tubes so as to be more easily able to ensure a good seal between envelope layer and tubes.

A suitable material for the material piece 11 is RTV (Room Temperature Vulcanizing) silicone system, which is addition-curing and which can be cross-linked at moderate temperatures. Such RTV silicones can be made sufficiently hard, yet are still pliable against the surface of the skin and self-adhesive. As can be seen below, the material piece 11 can also be produced in situ with a silicone composition which is highly viscous when applied to the plastics film 2 or directly to the skin, yet which then hardens at room temperature into a suitably hard and skin-friendly silicone elastomer. The tubes are then pressed carefully down in the silicone composition before this has hardened. A silicone composition of this type is known by virtue of WO 2004/108175 A1, to which reference should be made for closer details. One advantage of producing the material piece in this way is that, if applied directly to the skin in the highly viscous state, it is guaranteed to be able to penetrate into all irregularities and cracks in the skin, so that a very secure sealing function is obtained.

An expedient way of applying the material piece in situ is to utilize a twin-chambered plunger-type injector provided with a mixing nozzle in which the components of the silicone composition are stored. The two reactive silicone pre-polymers can thereby be kept separated and unreacted right until the components are pressed out through the nozzle. This twin-component addition-curing system can also be provided ready-mixed, if a sufficient quantity of inhibitor is added. Such a ready-mix has a limited usage time before it spontaneously cross-links and must be stored cold to prevent premature hardening.

By the term “highly viscous” is meant in this publication silicone compositions having a viscosity within the range 10-1000 Pa×s at the time of application, preferably 100-500 Pa×s. Viscosity and hardness after hardening can be controlled by the mix ratio of the two components in the silicone composition and by the addition of filling agent.

The shape of the material piece may vary, but it is important that the bottom side is plane and that the edges of the material piece are very thin, so that a load upon the edges is transmitted to the support surface as an areal load and not as a linear load. Moreover, the top side of the material piece preferably has a rounded shape. The term “plane” means, of course, viewed on a macroscale, since it is desirable for the material piece to force its way into all irregularities in the support surface, which in certain applications can be constituted by the skin of the patient, so as to ensure seal-tightness.

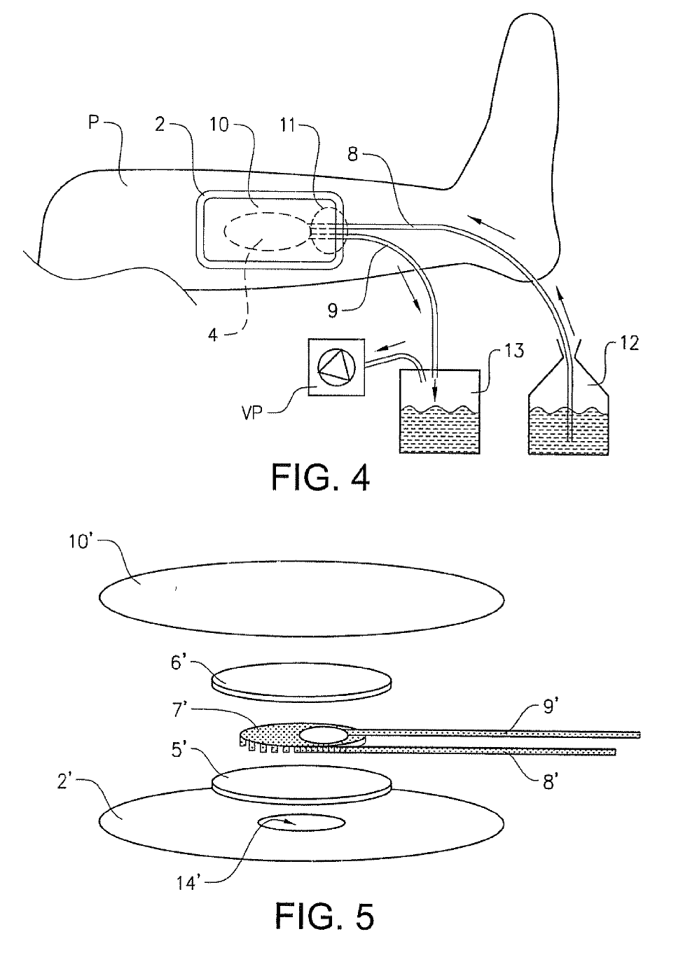

FIG. 4 shows diagrammatically the dressing in FIG. 1 applied to the leg of a patient P. The tube 8 is connected to a container 12 for irrigation fluid, and the tube 9 to a vacuum pump VP via a collecting vessel 13.

The dressing functions as follows.

When an underpressure is generated in the tube 9 with the aid of the pump VP, fluid present in the dressing will be sucked into the tube 9, which means that an underpressure is also generated in the tube 8. Irrigation fluid from the container 12 will then flow into the dressing and be distributed in that cellular body 5 of the two cellular bodies 5, 6 which is situated closest to the wound, and will flow over the wound bed on its way to the periphery of the membrane 7. The irrigation fluid mixed with excess exudate from the wound bed will then flow around the periphery of the membrane 7 and be sucked into the second cellular body 6 situated on the opposite side of the membrane from the first cellular body 5, and then further into the tube 9.

The container 12 of irrigation fluid is preferably closed against the surrounding atmosphere and has atmospheric pressure when start-up takes place. Moreover, the container 12 is preferably formed from a ductile material, so that it can be compressed by the atmospheric pressure as the irrigation fluid is sucked out of the container. Another possibility is to make the container be open to the atmosphere and in this case be expediently provided with a filter which prevents airborne bacteria or other harmful agents from accompanying the air into the container. In the preferred embodiment of the invention, the vacuum pump VP acts as a vacuum tank, i.e. there is no continuous activation of the pump, but rather the underpressure in the container 13 is reduced as liquid is filled. The flow through the dressing will cease once the pressure has fallen sufficiently far in the irrigation fluid container 12 that the pressure difference between the underpressure in the vessel 13 and the pressure (approximately atmospheric pressure) in the container 12 has been levelled out. The generated underpressure and the flow resistance in the system are dimensioned such that the flow velocity is low, preferably 0.5-100 ml per hour. This means that the fluid fed through the tube 8 trickles rather than flows into the space between the membrane 7 and the cellular body 5 and that correspondingly little quantity of fluid, together with pus secreted from the wound, is sucked up around the periphery of the membrane 7 and into the space above the membrane so as then to be evacuated through the tube 9. The low flow velocity means that the supplied fluid in the space beneath the membrane has a relatively long retention time in this space, which means that it can easily be spread into the cellular body and reach the wound bed itself. The low flow velocity within the dressing also serves to ensure that supplied liquid has time to force its way down towards the wound bed before it reaches the periphery of the membrane.

It is, of course, possible, by reactivating the pump VP, to produce a new underpressure when the pressure difference between the vessel 13 and the container 12 becomes too small, but the fact that the irrigation fluid passes so slowly through the dressing allows the fluid flow to continue for a period of at least eight hours without the pump needing to be reactivated. This allows the use of disposable-type vacuum pumps which, following use, can be discarded together with the dressing, for example the use of manual bellows pumps. In one advantageous variant, vacuum chambers of the bellows pump are utilized in such an application as storage vessels for sucked-up fluid, i.e, the tube 9 in FIG. 14 emerges directly into the vacuum chamber of a bellows pump. In such an application, underpressure and dressing are expediently dimensioned such that a fluid flow continues throughout the action period for the dressing, i.e. throughout the period for which the dressing must stay on. The irrigation process then takes place fully automatically following application of the dressing, without the need for any measures to be taken by staff or patients.

Moreover, it is then possible to connect the tube 9, instead of to a pump, to a vacuum tank in which a specific underpressure prevails. The vacuum tank can also be constituted by a pre-evacuated bellows pump, or a bellows pump having markings or limit stops, which mark out the intended compression of the pump in order to obtain a specific underpressure. If a vacuum tank, pre-evacuated bellows pump or the like is used the connection of the tube 9 to the vacuum chamber, i.e. the space which is put under underpressure, is expediently realized by holes being made in a membrane or the like which closes off the vacuum chamber prior to use, for example as the tube 9 is connected up or by rotary or other movement of the tube if the dressing, together with irrigation fluid container and vacuum source, is constructed as an integrated unit.

In one variant, the supply tube is provided with a valve, which allows the tube to be closed off, so that the irrigation liquid container can be exchanged without losing the underpressure in the wound bed. An empty irrigation liquid container can hence be replaced by a new one, or a container holding a type of irrigation liquid can also be replaced by another container holding a different type of irrigation liquid. It is also possible, of course, to vary the underpressure by compressing the bellows pump if the pressure has fallen too far or if, for some other reason, it is wished to increase the underpressure. If a vacuum tank is used in the dressing instead of a bellows pump, this can expediently be provided with a shut-off valve for allowing a change of dressing without remaining underpressure in the container being lost.

It is pointed out that, in order for a dressing according to the above-described preferred embodiment to function in the above-described manner, the adhesive coating 3 and the material piece 11 must ensure sealing against microleakage of air throughout the period of action of the dressing. It is also pointed out that the dressing must also, of course, be seal-tight with respect to macroleakage. If the skin were to have unusually deep cracks, it might therefore be necessary to seal such cracks before the dressing is applied. This is expediently done with a silicone composition which is highly viscous when applied to the skin, and thus fills such cracks, and which then hardens at room temperature into a soft and skin-friendly silicone elastomer. Such a silicone composition is known by virtue of WO 2004/108175 A1. The irrigation fluid can advantageously contain agents which facilitate healing or are otherwise beneficial, for example antibiotics, antimicrobial agents, antiseptics, growth factors, pH buffer (for example acidification), salts (for example physical NaCl solution), antioxidants, vitamins, enzymes (for example proteolytic enzymes), nutrition substances.

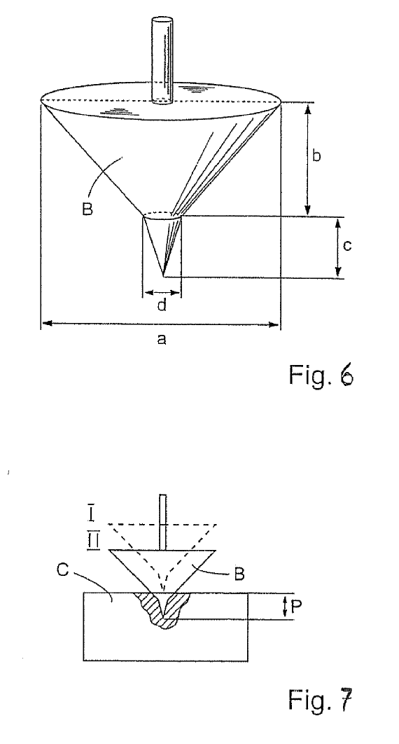

In FIG. 5, a second embodiment of the invention is shown, which differs from the embodiment shown in FIGS. 1 and 4 primarily by the fact that the dressing in FIG. 5 has a somewhat differently configured membrane 7′. Those components in the dressing which correspond to like components in the embodiment shown in FIG. 1 have been given the same reference symbols with the addition of a prime sign.

The film 2′, like the film 2 in the embodiment shown in FIG. 1, is provided with a coating of a soft skin-friendly adhesive, which seals against microleakage (not shown in FIG. 4). In its central part, the film 2′ has a cutout 14, which at least extends around the wound bed and leaves this clear. The component 4′, consisting of bodies 5′, 6′ of liquid-permeable material, the fluid-tight membrane 7′ and the tubes 8′, 9′ differ from corresponding components in FIG. 1 primarily by the fact that the membrane 7′ is provided with a pattern of protrusions, which extend on the bottom side of the membrane 7′. The protrusions have in FIG. 5 a cylindrical shape, but can have any shape whatsoever, including a linear shape. The purpose of the protrusions is to ensure that fluid can be distributed over the entire surface of an underlying material body, even if this is locally clogged, for example by particles or the like from the wound bed. In order to achieve this, it is not always necessary to form protrusions, but rather it may be sufficient to choose for the membrane 7′ a material having an uneven surface structure, for example a textile material or non-woven.

Moreover, the ends of the tubes 8′, 9′ are integrally connected to the membrane, so that membrane and tubes can be applied as a unit. The material bodies 5′, 6′, too, can be connected to the membrane.

The dressing shown in FIG. 5, like the dressing shown in FIG. 1, can be provided having all the components assembled into a unit and also then comprises a pressure-distributing material piece, which encloses parts of the tubes 8′, 9′ but can also be provided having the components 2′, 4′ and 10′ configured as separate units. The layers of adhesive on the envelope layer 10′ and the film 2′ are in this case covered by a release layer, for example a polyethylene film, and a stiffening layer is detachably fixed to the top sides of the films. Such a configuration allows a precise adaptation of the dressing to the shape of the wound to which it is to be applied. Such a dressing is applied as follows:

a) first a central hole, corresponding to the contour of the wound bed, is cut out of the plastics film 2′ coated with a skin-friendly, sealing adhesive, and the plastics film 2′ is fixed to the skin around the wound bed (the release layer is removed preferably after cutting to size), whereafter:
b) a highly viscous silicone composition of the abovementioned type is applied to a part of the plastics film 2′ extending from the edge of the hole 13 and out to the periphery of the plastics film 2′,
c) the unit 4′ comprising a first body 5′ of soft liquid-permeable material, an airtight and liquid-tight membrane 7′ and tubes 8′, 9′ for the supply and evacuation of fluid, of which the fluid supply tube 8′ emerges on one side of the membrane and the fluid evacuation tube or tubes 9′ on the other side of the membrane, is applied to the wound bed with the first material body 5′ closest to the wound bed, so that the said holes are fully covered by the first material body, so that the tubes extend in the region of the applied viscous silicone composition and the tubes are pressed carefully down in the composition, whereafter
d) the envelope layer 10′ of flexible material is applied on top of the said unit and is sealingly fixed to the plastics film, whereafter the silicone composition enclosing the tubes is allowed to harden for a few minutes, whereafter
e) the tubes for the supply and evacuation of fluid are connected to a container for irrigation liquid and a vacuum pump, respectively, before or after the measures a-d.

In one embodiment of the dressing (not shown), there is no lower film (the film 2, 2′ in the illustrated embodiments) and the soft and skin-friendly adhesive is directly fixed to the bottom side of the envelope layer. In this embodiment, too, the envelope layer constitutes a first unit, which is separate from a second unit consisting of cellular bodies, membrane and tubes. Preferably, the second unit is also a physical unit, i.e. the constituent components are fixed to one another. Such a dressing is applied as follows:

a) the second unit consisting of cellular bodies, membrane and tubes is cut to size so that it acquires a contour corresponding to the contour of the wound bed (of course, without the tubes being severed), whereafter
b) a highly viscous silicone composition of the abovementioned type is applied directly to the skin extending from the wound edge in the direction away from the wound bed, whereafter
c) the second unit is applied to the wound bed with the first cellular body closest to the wound bed and so that the tubes are situated in the applied viscous silicone composition and the tubes are then pressed carefully down in this composition, whereafter
d) the envelope layer coated with a layer of adhesive is applied on top of the second unit and is sealingly fixed to the skin around the wound bed, whereafter the silicone composition is allowed to harden for about a minute,
e) the tubes for the supply and evacuation of fluid are connected to a container for irrigation liquid and a vacuum pump, respectively, before or after the measures a-d.

If the skin around the wound has “macrocracks”, these are expediently filled with the aforesaid highly viscous silicone composition, which is then allowed to harden in situ. The dressing must then be applied before such a composition has clearly hardened.

If the material piece 11 is applied in situ in the above-stated manner, the silicone composition will be adherent to the above-lying envelope layer and underlying skin or plastics film, so that no further glue or the like for fixing the material piece to the plastics film or the skin is needed to ensure seal-tightness.

Moreover, the silicone composition can then, of course, be applied so that it extends beyond the plastics film and envelope layer, if so desired.

In the embodiment shown in FIG. 1, a perforated layer of adhesive 3 is applied next to the wound bed. In those embodiments in which the adhesive layer 3 is only applied beyond the wound bed, it is expedient to coat the cellular body 5, 5′ with a layer which is not adherent to the wound bed. Such a layer must, of course, be liquid-permeable and can be constituted by a reticular layer, a perforated layer, or a layer which, for example, has been sprayed over the entire surface of the cellular body. Those adhesives which are suitable for the adhesive layer 3 can also be used to produce layers which do not adhere to the wound bed. However, they do not, of course, need to have the softness or weight per unit area which is necessary for the layer 3, which must secure against microleakage.

As is indicated in FIGS. 4 and 5, the membrane 7, 7′ and the tubes 8, 9, 8′,9′ expediently constitute an integral unit, which, for example, can be moulded in one piece, or can otherwise be assembled into a physical unit.

The envelope layer 10, 10′ is constituted by a thin and very flexible material, for example a polyurethane film with a thickness less than 50 micrometres. Other plastics materials may also be used.

The film 2, 2′, also, is constituted by a thin, soft and pliable material, for example polyurethane with a thickness less than 50 micrometres. In the illustrated embodiments, the material body 5, 5′ is constituted by a soft, open-celled polyurethane foam. It is conceivable, however, to use other liquid-permeable materials which can be formed such that they can adapt their shape to the shape of the wound bed so that they come to bear against the latter or against any underlying perforated film layer. Examples of such materials are cotton wool, non-woven and textile material. The material used is preferably hydrophilic.

The membrane can be constituted by a thin sheet of rubber of plastics material, for example polyurethane film with a thickness of 25-50 micrometres. It is also conceivable to use a liquid-tight and airtight textile material, or laminate of one or more textile materials and one or more plastics materials. It is pointed out that the thickness relates to wall thickness and not to the thickness of the membrane inclusive of any protrusions. If the height of the protrusions shown in FIG. 15 is taken into account, the membrane can have a thickness of 1-3 mm.

The material body 6, 6′ can be constituted by any one of the materials which are suitable for the body 5, 5′. The foremost function of the body 6, 6′ is to prevent the opening of the tube 9, or the inlet thereto on the top side of the membrane 7, 7′, from being shut off by the envelope layer 10, 10′ being pressed against the membrane owing to the pressure difference between the atmosphere outside the dressing and the underpressure inside the dressing. It is therefore, for example, conceivable to replace the material body 6, 6′ with a sheet of ductile material, which, on its side facing the membrane, has a peripheral annular duct and a number of ducts which lead to the mouth of the tube 9.

The tubes are constituted by polyurethane, silicone or other commonly found tube materials for medical products. The internal diameter for the evacuation tube 9, 9′ is 0.15-2 mm and for the supply tube 8, 8′ 1-10 mm. It shall be noted that the evacuation tube preferably has a greater internal diameter than the supply tube.

It is pointed out that even though the tubes in the figures have been shown extending from membrane to pump and irrigation liquid container respectively, the tubes can be constituted by a plurality of interconnectable parts. Moreover, a tube-connecting part can be connected to the membrane, for example configured in one piece with the membrane, the tubes being joined together with the connecting part in connection with the application.

A dressing according to the embodiment in FIG. 1 has been tested together with a Bellovac pump (bellows pump). The dressing was applied to a test plate of the same type as in an MHC leakage test with a groove depth of 75 micrometres. The bellows pump was then compressed so that an underpressure of 18.67 kPa (140 mm Hg) was created in the evacuation tube, whereafter irrigation liquid was allowed to flow through the dressing. The mean flow velocity of the irrigation liquid was about 4.5 ml/hour. After 24 hours, the underpressure had been reduced by 25-40%. The test reveals that the dressing was seal-tight, so that no air leaked in, but rather the pressure reduction was due merely to the transfer of liquid from the irrigation container.

A dressing according to the embodiment in FIG. 1 has also been tested fixed to a person for 4 and 6 hours respectively. In this case, too, the pressure reduction was small, about 10%.

The products referred to in the present invention are normally supplied sterile-packed, which means that the adhesives used must be sterilizable, like also, of course, other components in the dressing itself, such as tubes.

Above, the invention has been applied to an irrigation dressing having at least two tubes, one for the supply of irrigation fluid and one for the evacuation of irrigation fluid. The invention can, of course, be applied in dressings having only one tube, which alternately supplies irrigation liquid to the wound bed and evacuates irrigation liquid and wound exudates from the wound bed, or in so-called vacuum dressings, in which only wound exudate is evacuated with the aid of underpressure, or other dressings, in which a tube or tubes lead(s) out from a wound or the like.

The described embodiments can, of course, be modified within the scope of the invention. For example, more than one connection for the supply of irrigation fluid, and/or more than one connection for the evacuation of irrigation fluid, can be included in the dressing. Moreover, the tubes do not need to run alongside each other, even though this is preferred, but can run in different directions and leave the dressing at several points along the periphery of the envelope layer or plastics film, each tube being enclosed in a pressure-distributing material piece. The tubes leading to the pump and irrigation fluid container, respectively, can be configured with a plurality of interconnectable parts, and such a connection in a tube part disposed next to the vacuum source can be such that a seal on this tube part is broken as the parts are joined together, for example by the piercing of a membrane in the tube end. The irrigation fluid container can be of the bag type or bottle type. If the irrigation fluid container is of the bottle type, it is open to the atmosphere and is preferably provided with a filter against airborne impurities. The dressing can contain a separate tube or the like for the supply or wound treatment agents. Such a tube can extend to the bottom side of the membrane or can constitute a branch line to the supply tube. The supply and evacuation tubes, at least close to the dressing itself, can be integrated in a single unit, i.e. a tube, which is divided into two ducts by a transverse wall. It is also conceivable to configure such a unit, or a part of occurring tubes, with a portion having the same cross-sectional shape as the material piece 11 in FIG. 3, and hence to make the material piece an integral part of the tubes. In such an application, this part is provided with a suitable adhesive for sealing against the support surface. The scope of the invention shall therefore only be determined by the content of the appended patent claims.

1. Wound dressing comprising a tube (9; 9′) for evacuating fluid from a wound and an air-tight and liquid-tight envelope layer (10; 10′) of flexible material, which envelope layer has a central region, in which the tube emerges beneath the envelope layer, and a peripheral region, which extends beyond the central region, the tube from the central region running outwards into the peripheral region beneath the envelope layer and out from this, characterized in that the tube (9; 9′) is enclosed in a pressure-distributing material piece (11) which surrounds the tube and has a hardness such that, when subjected to an external load, the forces will be distributed in the material piece and will bring load to bear upon underlying skin and tissue spread over a relatively large plane surface, and which extends along the tube in the peripheral region and also extends laterally beyond the tube viewed in the longitudinal direction of the tube.

2. Wound dressing according to claim 1, in which the pressure-distributing material piece (11) has in cross section a shape which tapers off from the tube in the lateral direction.

3. Wound dressing according to claim 2, in which the pressure-distributing material piece (11) consists of a silicone composition.

4. Wound dressing according to claim 3, in which the silicone composition consists of an addition-curing RTV silicone system.

5. Wound dressing according to claim 4, in which the material piece (11) is adherent to skin.

6. Wound dressing according to claim 5, in which the material piece (11) is shaped in situ.

7. Wound dressing according to claim 1, in which a tube (8; 8′) is provided for the supply of fluid to a wound and is enclosed in a pressure-distributing material piece (11).

8. Wound dressing according to claim 7, in which the tubes (8, 9; 8′, 9′) for the evacuation and supply of fluid run alongside each other and are enclosed in the same material piece (11).

9. Wound dressing according to claim 2, in which a tube (8; 8′) is provided for the supply of fluid to a wound and is enclosed in a pressure-distributing material piece (11).

10. Wound dressing according to claim 3, in which a tube (8; 8′) is provided for the supply of fluid to a wound and is enclosed in a pressure-distributing material piece (11).

11. Wound dressing according to claim 4, in which a tube (8; 8′) is provided for the supply of fluid to a wound and is enclosed in a pressure-distributing material piece (11).

12. Wound dressing according to claim 5, in which a tube (8; 8′) is provided for the supply of fluid to a wound and is enclosed in a pressure-distributing material piece (11).

13. Wound dressing according to claim 6, in which a tube (8; 8′) is provided for the supply of fluid to a wound and is enclosed in a pressure-distributing material piece (11).
